(19) United States
(12) Patent Application Publication
(10) Pub. No.: US 2015/0209173 A1
(43) Pub. Date: Jul. 30, 2015

(54) Device for a Temporary Ileostomy or Urostomy Closure

(71) Applicant: Peter Kratky, Starnberg (DE)

(72) Inventor: Peter Kratky, Starnberg (DE)

(21) Appl. No.: 14/678,442

(22) Filed: Apr. 3, 2015

Related U.S. Application Data
(63) Continuation-in-part of application No. 12/809,648, filed on Jan. 14, 2011, now abandoned.

(51) Int. Cl. A61F 5/445

(52) U.S. Cl. CPC A61F 5/445

(57) ABSTRACT

A device for a temporary seal of a stoma coupled to an intestine having an intestinal lumen includes a base plate is configured to be applied permanently about a periphery of the stoma on an outside of a body. A cover is releasably connected to a base plate for forming a temporary stoma steal and that is configured in such a way that it forms a volume for receiving at least one receptacle for fluid emerging from the stoma. A tube-shaped container is detachably coupled with the base plate and disposed within the cover, but not attached to the cover so, the container configured to be separable from the base plate. The container opens to the stoma and configured to receive fluid emerging therefrom.

### CROSS-REFERENCE TO RELATED APPLICATION(S)

This application is a continuation-in-part of, and claims the benefit of, U.S. patent application Ser. No. 12/809,648, filed Jan. 14, 2011, the entirety of which is hereby incorporated herein by reference.

This application claims the benefit of DE Patent Application No. 10 2007 062 133.9, filed Dec. 21, 2007, the entirety of which is hereby incorporated herein by reference.

### BACKGROUND OF THE INVENTION

1. Field of the Invention

The present invention relates to a device for a temporary ileostomy or urostomy closure.

2. Background of the Invention

A device of the type mentioned above is used in persons having a surgically made opening of a hollow organ toward the body surface, e.g. an artificial anus, a so-called stoma. The word stoma is derived from Greek and translates as “orifice” or “opening”. Adequate prefixes enable differentiation of various bowel outlets. Diversion of the small intestine is called ileostomy, diversion of the large intestine is called colostomy, and diversion of urine is called urostomy. Colostomy is further differentiated into descendostomy or sigmoidostomy and transversostomy, urostomy being differentiated into a urethral fistula and a conduit.

A stoma can be applied provisionally (temporarily) or on a permanent basis (permanently). A stoma can be a single-lumen (on the end or terminal) or double-lumen (double-barreled) stoma.

The most frequent cause for applying a stoma is cancer of the rectum (rectal carcinoma) or the anus (anal carcinoma). During surgical removal of the tumor, it may be necessary in addition for the bowel sphincter to be removed and for an artificial anus to be made on the abdomen.

In some surgical techniques, the natural sphincter may be preserved under certain circumstances. In this case, only a provisional artificial anus is applied in order to ensure for the suture site on the large intestine to heal (anastomotic protection).

The second most frequent cause is a medical condition of a chronic inflammatory bowel disease, such as Crohn's disease or ulcerative colitis. In these medical conditions, application of a stoma allows e.g. for inflammatory intestinal segments to be immobilized for some time or on a permanent basis. If the diseased bowel has to be removed in part or in total, application of a stoma may also be required.

For an Anus praeter of the ileum, liquid aggressive intestinal contents are evacuated via the stoma. Depending on drinking and eating habits, the volume of liquid amounts to about 2500 ml/day. Terminal and double-barreled ileostomy is differentiated. The type applied depends on the medical condition.

For an Anus praeter of the large intestine, more or less shaped stool evacuates via the colostomy. Colostomies imply strong gas formation.

As in ileostomy, a differentiation is made between terminal and double-barreled colostomy: terminal stoma application is always applied if there is a medical condition in the rectum or at the anus requiring removal of the rectum, anus together with the sphincter, or partial removal of the diseased intestinal segment, and if the new enteroanastomosis can only be performed after a time interval of several weeks or months. A permanently remaining ileostomy is required for instance subsequently to proctocolectomy if restoration by means of anastomosis with the straight intestine is no longer possible for technical or functional reasons. Other disorders require provisional immobilization of the intestinal segment involved, so that upon healing of the same, the stoma can be returned. In the case of a urostomy, the stoma is created for the diversion of urine. Due to various medical conditions of the urine discharge system, functionality thereof is lost. In order to be able to ensure this functionality once again, proven surgical techniques are required which in general result in artificial urine discharge. The most frequent forms of urine discharge are the ileum conduit, the colon conduit, and on rare occasions also urethral fistulae.

There are dry and wet urostomies. In the wet stoma, such as in the urethral fistula, the ureter remote from the bladder is pulled directly through the abdominal wall and fastened to the skin. In most cases, the ureter has to remain splinted on a permanent basis by means of a thin catheter since the stoma tends to narrow and thus to hinder drainage.

In the case of an ileum conduit, the remote ureter is implanted into a neutralized part of the small intestine. This segment is sewed into the abdominal wall in the form of a nipple. In the case of a colon conduit, remote ureters are implanted into a neutralized part of the large intestine. This part is also sewed into the abdominal wall in the form of a nipple.

As alternative surgical techniques for continent ileostomy, the ileoanal pouch (J, S, or W pouch) and the Kock pouch should be mentioned. Said surgical options may not be possible in every case. In the ileoanal pouch, as an alternative to an ileostomy, upon removal of the large intestine, an artificial reservoir similar to a pocket is produced from the last loops of the small intestine. At the lower end, this reservoir has an opening which is connected to the sphincter tract. With the sphincter preserved, stool then accumulates inside the pouch and may subsequently be egested via the anus as before. In severe cases of ulcerative colitis, the pouch is usually applied by very experienced surgeons as a J pouch.

In the case of the Kock pouch, an artificial reservoir similar to a pouch is also formed from the lower loops of the small intestine. Another piece of intestine is withdrawn from the small intestine and used as a connection from the Kock pouch to a small opening in the abdominal wall. The surgical technique ensures that during the filling phase of the reservoir stool cannot reach the outside through the opening in the abdominal wall, the reservoir being emptied only by the introduction of a catheter. Nowadays, the ileoanal pouch is preferred over the Kock pouch as the method of choice.

It is estimated that in the Western world, out of 1000 persons, one is living with a stoma.

Usually, small intestine stomata (ileostomy) and urostomies must be discharged into a stoma pouch. Depending on food supply, the liquid intestinal contents in small intestine diversions amount to up to 2500 ml/day. The large intestine is neutralized and no longer performs its normal function (regulation of water and salt balance). For the patient involved, this means that he or she must constantly carry a stoma pouch on the body, into which on average about 80 to 100 ml of liquid will drain off every hour in case of ileostomy. The amount of liquid may be regulated by appropriate drinking and eating behavior, but it must not exceed certain amounts on a daily average in order to avoid interferences of the water balance and thus among others also renal dysfunction (loss of liquid and electrolytes).

In addition to carcinogenic medical conditions of the colon, inflammatory medical conditions of the large and small intestine (e.g. sigma diverticulitis, Crohn's disease, or ulcerative colitis) may also result in the necessity of applying a small intestine stoma. In particular, patients with medical conditions from the fields of Crohn's disease or ulcerative colitis quite frequently have to receive a temporary ileostomy or frequently a permanent ileostomy. This is required in order to bring about healing of persistent formation of fistulae in the rectal area or in case of improper sphincter function. Patients with inflammatory intestinal medical conditions are mostly young patients and, in the relevant case, will have to live with an ileostomy for many years.

The indication for the application of a urostomy (ileum conduit or Bricker bladder) is among others given in the presence of bladder ureter tumors, atresias, and malformations, contracted bladder (radiation damage), gynecological tumors, neurogenic bladder diversion dysfunctions, tumors of the lesser pelvis, and injuries.

Irrigation is a term from Greek and means “watering” or “rinsing”. Such rinsing of the intestine in regular intervals causes regular emptying thereof. After irrigation, no excretion is to be expected for 24 to 48 hours so that during this time no pouch, but only an unobtrusive stoma cap can be carried. However, irrigation is only appropriate for stoma carriers with solidly shaped excretions, and even then, only under certain conditions. For irrigation, a special irrigation set is required.

3. State of the Art

For the protection of the various stomata during certain activities, the industry has developed a whole range of different styled stoma bandages or stoma girdles for men and women. In such bandages and girdles, usually a pocket for the pouch is integrated and is to provide the patient with more safety during leisure, sports, and work.

The product offer for stoma treatment is almost unbelievably large. Most of the time, the products used provide a single-piece treatment, i.e. the skin protection plate is integral with the pouch. In order to avoid overstraining of the skin, single-piece units should be replaced no more than three times a day. Single-piece units are very flat, protrude very little, are flexible and easy to use.

The two-piece treatment includes a base plate as skin protection and an associated pouch. A snap-in ring is attached to the base plate into which the pouch can be snapped easily. The base plate may stay on the skin for several days, but the pouch can still be replaced as many times as desired.

As pouch types, there are closed pouches which are in general used for colostomy. In general, closed pouches have an activated charcoal filter which allows for gases produced during digestion to escape without odor. As appropriate, the closed pouch can be replaced about two or three times a day.

Smoothing pouches (open pouches) are mostly used for ileostomy as it will mainly produce liquid to pasty excretions. Ileostomy pouches have a bottom outlet which is closed by a clasp so that the person involved can empty the pouch via the bottom outlet and does not have to constantly replace the entire treatment. In most cases, these are pouches without integrated filters; however, several manufacturers also offer smoothing pouches with integrated filters.

Due to the large liquid volume produced, stoma carriers with small intestine stomata/urostomies are obliged to carry a permanent pouch treatment.

Devices and apparatus for a temporary colostomy closure are known as such (e.g. from U.S. Pat. No. 6,050,982 and WO90/07311). However, use of such stoma caps, as are implemented upon irrigation in the case of colostomy, and which thus represent a limited time “liberation” of a stoma pouch, are not possible for ileostomy or urostomy carriers for technical reasons. In the case of an ileostomy or urostomy, large volumes of liquid are produced, for which a conventional colostomy closure is neither appropriate nor designed. For this purpose, only the known stoma pouches are used. At the present time, the ileostomy or urostomy carriers involved have to put up with a stoma pouch in their life. This applies exceedingly to young patients suffering from inflammatory intestinal medical conditions.

### SUMMARY OF THE INVENTION

An object of the invention is in particular to create an economical and easy-to-use device enabling a person involved with an ileostomy or urostomy treatment to go without the required pouch treatment for a certain time.

In one aspect, the invention includes a device for a temporary seal of a stoma coupled to an intestine having an intestinal lumen. A base plate is configured to be applied permanently about a periphery of the stoma on an outside of a body. A cover is releasably connected to a base plate for forming a temporary stoma steal and that is configured in such a way that it forms a volume for receiving at least one receptacle for fluid emerging from the stoma. A tube-shaped container is detachably coupled with the base plate and disposed within the cover, but not attached to the cover so, the container configured to be separable from the base plate. The container opens to the stoma and configured to receive fluid emerging therefrom.

In another aspect, the invention includes a device for a temporary ileostomy or urostomy closure. A base plate is to be permanently attached in the periphery of a stoma on the outside of a body surface. A cover that releasably connected to the base plate for forming a temporary stoma closure. On the inner side of the cover, a tube member protrudes into the stoma is fastened, and wherein a chamber is made around the tube member, which is configured for receiving contents emerging from the stoma through an axial main channel of the protruding tube member.

Further aspects of the invention will result from the respective dependent claims, the description below of sample embodiments, and the appended drawings.

### BRIEF DESCRIPTION OF THE FIGURES OF THE DRAWINGS

Further embodiments of the invention will now be described by way of example only and with reference to the appended drawing. In the drawing:

FIG. 1 schematically shows a terminal ileostomy;

FIG. 2 schematically shows a double-barreled ileostomy;

FIG. 3 schematically shows a colon conduit;

FIG. 4 schematically shows an ileum conduit;

FIG. 5 schematically shows a schematic cross-sectional view of the inventive arrangement with a closing cap and a cover plate on a base plate;

FIG. 6 schematically shows a schematic illustration of an inventive device from above;

FIG. 7 schematically shows a schematic cross-sectional view of an inventive device with an outer and an inner film container;

FIG. 8 schematically shows a schematic cross-sectional view of an inventive device with the cover plate on the base plate with an insertion aid;

FIG. 9 schematically shows a schematic cross-sectional view of an inventive device with the cover plate on the base plate with a valve mechanism;

FIG. 10 schematically shows a schematic cross-sectional view of an inventive device with the cover plate on the base plate with an optionally fillable balloon;

FIG. 11 schematically shows a schematic cross-sectional view of an inventive device with the cover plate on the base plate with an umbrella;

FIG. 12 schematically shows a schematic cross-sectional view of an inventive device with the cover plate on the base plate with a split cylinder;

FIG. 13 schematically shows a schematic illustration of the cylinder of FIG. 12; and

FIG. 14 schematically shows a schematic illustration of an alternative sample embodiment of a device for a temporary ileostomy or urostomy closure.

The figures are not necessarily to scale, certain parts are only represented figuratively, the focus being on the principle of the invention.

### DETAILED DESCRIPTION OF THE INVENTION

Prior to a description of sample embodiments of the inventive device, several illustrations of various types of stomata will follow hereafter.

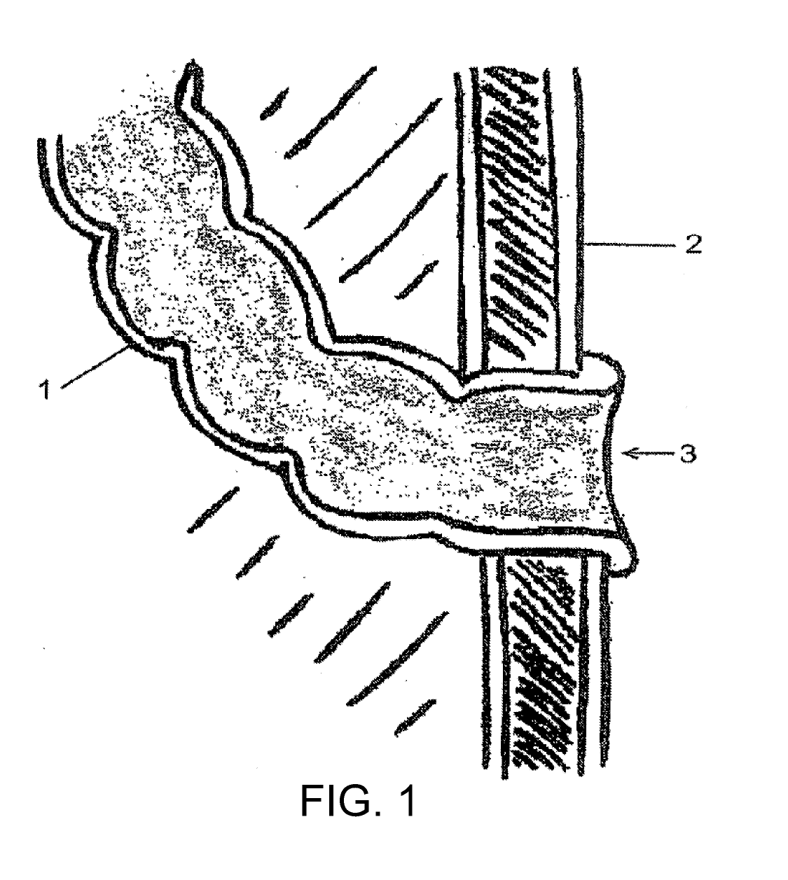

FIG. 1 shows a terminal ileostomy with the feeding intestine 1, which according to one sample embodiment is guided through a person's abdominal wall 2 forming a stoma 3 therein.

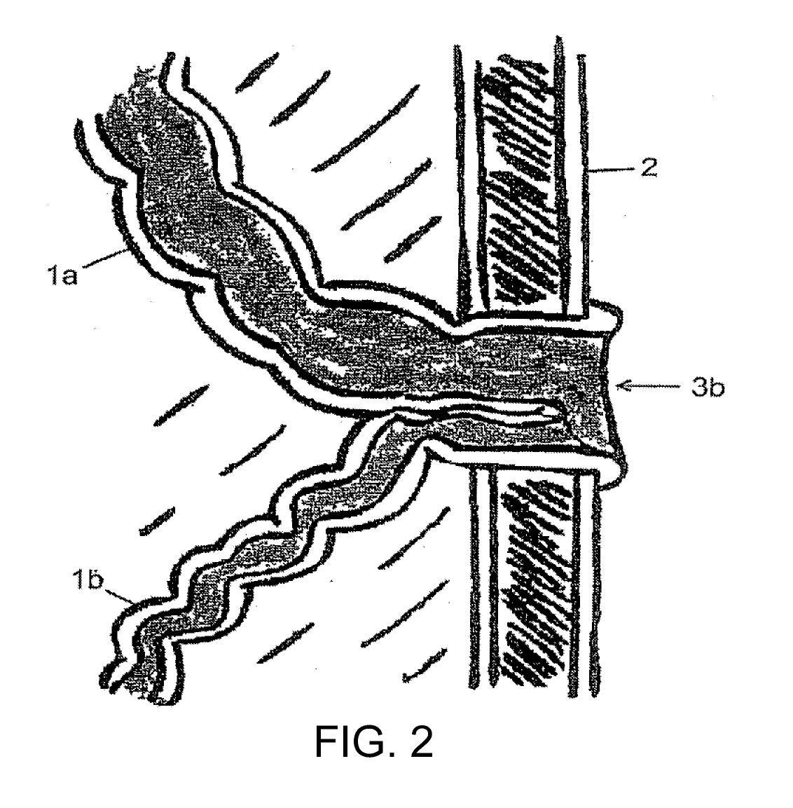

According to another sample embodiment, FIG. 2 shows a double-barreled ileostomy with a feeding bowel part 1a, an evacuating bowel part 1b, and the stoma 3 on the abdominal wall 2.

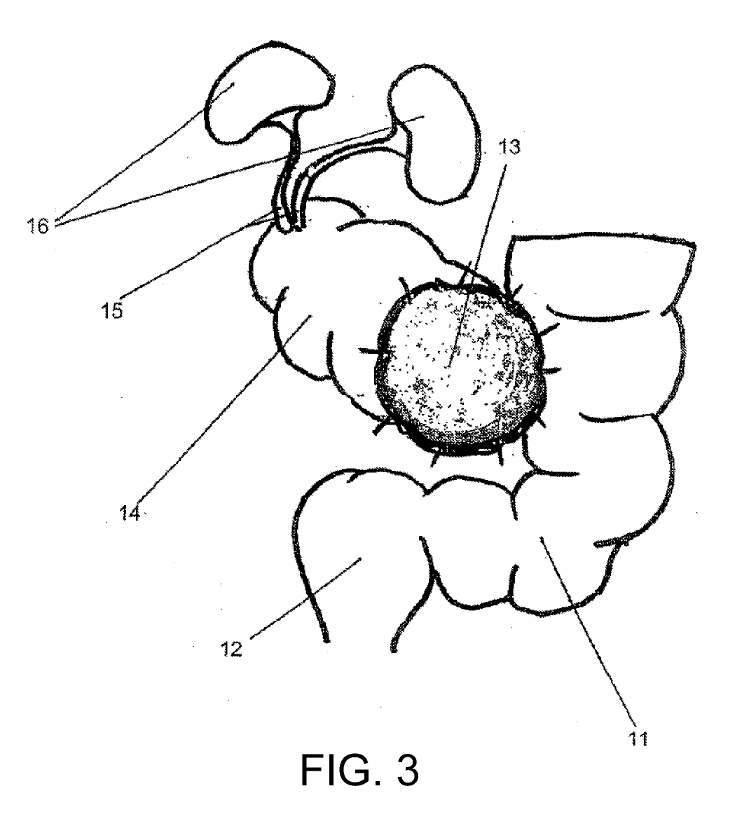

According to a further sample embodiment, FIG. 3 shows a colon conduit wherein the kidneys 16 are emptied via an implanted ureter 15 into a neutralized segment 14 of the large intestine. The segment 14 of the large intestine opens into a stoma 13 sewed into the abdominal wall (not shown). In the background, a colon (sigma) 11 and a rectum 12 are visible.

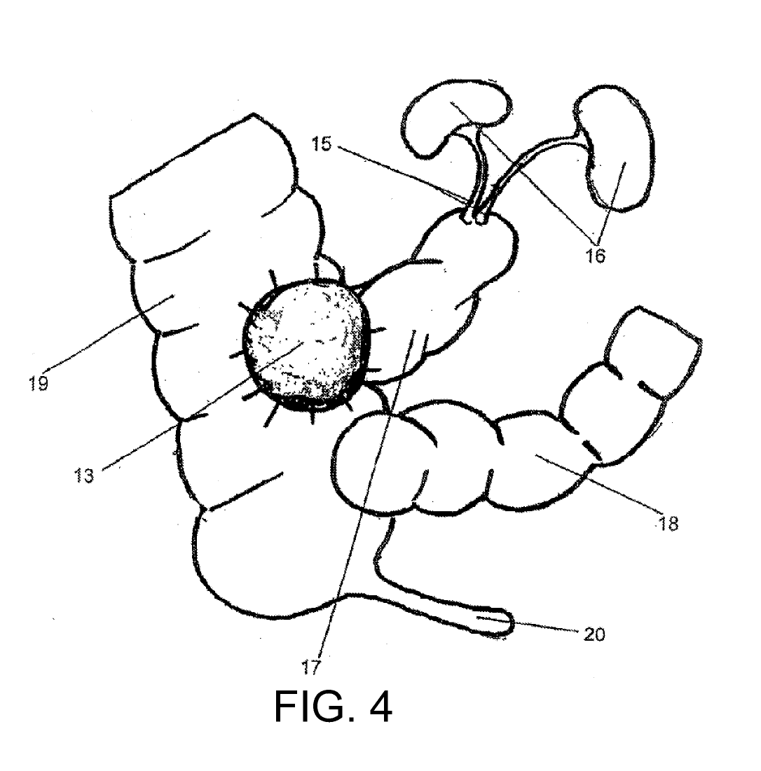

According to a further sample embodiment, FIG. 4 shows an ileum conduit wherein the kidneys 16 are emptied via the implanted ureter 15 into a neutralized segment 17 of the small intestine, the segment 17 of the small intestine opening into a stoma 13. In the background, a small intestine 18, an appendix 20, and a colon ascendens 19 are visible.

Hereafter, various sample embodiments of the inventive device for a temporary ileostomy or urostomy closure will be described more in detail. Herein, the term “stoma closure” refers to an ileostomy closure or a urostomy closure.

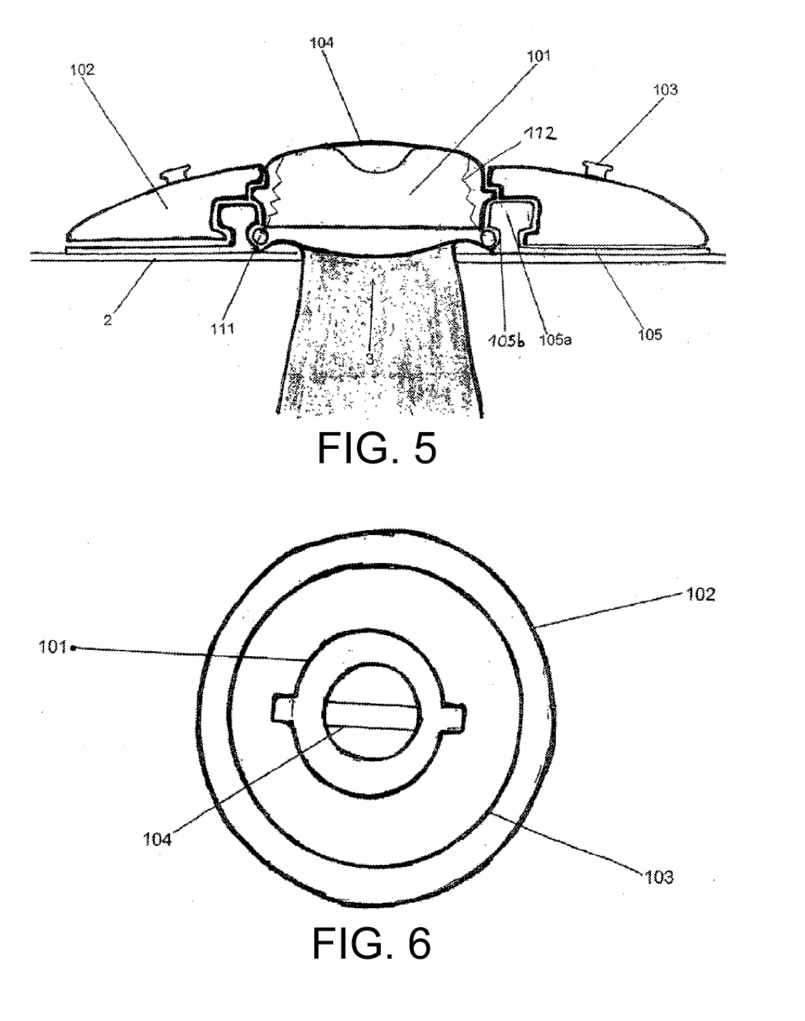

FIG. 5 shows a first sample embodiment of an inventive device in a schematic cross-sectional view. The inventive device is to temporarily replace a pouch known from prior art for receiving the contents emerging from the stoma. For this purpose, it comprises a base plate 105 made to be permanently attached in the periphery of a stoma on the outside of an abdominal wall 2. Moreover, a cover is releasably connected to the base plate 105 for forming a temporary stoma closure, and is shaped like a half-shell closing cap 101 so as to constitute a receiving volume for receiving at least one receptacle in the form of a folded container film 112, which will be described more in detail with reference to the following figures, for fluid emerging from the stoma 3. As can be seen FIG. 5, at the open end, the receptacle is releasably coupled to the base plate 105. However, the closing cap 101 can also be detached from the base plate independently from the receptacle. The base plate 105 is provided with a peripheral recess 105b into which the receptacle can be hung at the open end thereof.

On the base plate 105 with a circumferential snap-in ring 105a, which is attached to the cleaned and degreased abdominal wall 2, a flat cover plate 102 is fastened. This cover plate 102 can be designed as a reusable unit and can be coated with an odor-stopping means. Coating can be done with metal oxides known to be good catalysts. In the form of nanoscale particles, they can be incorporated into a sol-gel network and applied to the cover plate 102. In addition, a coating can be used to have a non-soiling effect (lotus effect). Variants of such coatings are already used in the automotive industry as “scratch-resistant paint”. In one sample embodiment, an electronic receiver unit for a level sensor can be accommodated in the cover plate. The location of the receiver unit is configured so as to allow for mechanical or other electronic contact with the sensor indicating the level of a diverted stoma/urostomy. In the middle, the cover plate 102 has a center opening for inserting the half-shell closing cap 101. The ventral surface of the cover plate 102 may have a snap-in ring 103. Upon removal of the closing cap 101, it is thus possible to optionally fasten an ordinary stoma pouch. Preferably, the cover plate and the closing cap are formed of a non-deformable or only slightly deformable material, e.g. plastic. In a further sample embodiment, the cover plate and the closing cap constitute an integral unit and thereby the inventive cover.

FIG. 6 shows a sample embodiment of an inventive device with a cover plate 102 in top view. The exchangeable, inside closing cap 101 can act as a pouch replacement. The closing cap 101 is for instance attached by means of a bayonet-type closure in the cover plate 102 by a push-down and rotating motion. For this purpose, there is a grip 104 on the closing cap. In an alternative embodiment, the cover plate and the closing cap can be made as one piece.

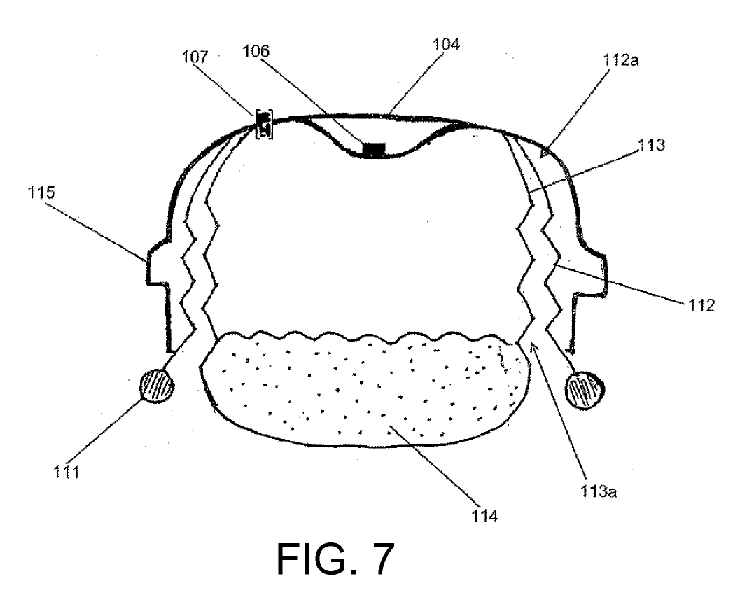

According to FIG. 7, the closing cap 101 according to one sample embodiment of the inventive cover can be embodied as a plastic half-shell inside which an accordion-pleated container film 112 is received. The container film 112 forms a first film container 112a, and in one variant of the sample embodiment, it may also be the only container film. This (first) film container 112a deploys upon release of the closing cap 101 from the base plate 105 (not shown), and when the cover has been removed, can thereby receive the liquid or semi-liquid intestinal contents emerging from the intestine. The inlet of the first film container 112a oriented towards the stoma 3 has a circular peripheral beaded ring 111 which is applied at the stoma outlet at skin level inside a recess on the base plate 105 or cover plate 102 sealing the same. In one embodiment, the first film container is made completely or partially of some metallic material. When the film container 112a has been filled with the intestinal contents, a torque motion on the closing cap is to achieve that the open end of the expanded film container, fixed inside the recess 105b of the base plate, is closed by the rotational motion, and that the contents cannot escape. Once the film container has been filled, due to brief fastening of the beaded ring 111 to the base plate 105, the torque motion will cause torsion of the film container. In case of a rotational motion of the flexible tube, such fastening causes torque closure of the metal part of the flexible tube. Then, the full film container can be removed from the base plate. Alternatively or additionally, for securing the contents, any of the commercially available disposable clasps can be placed thereon.

The closing cap may comprise a filter 107, in particular an activated charcoal filter, which allows for gases produced during digestion to escape without odor.

In a further embodiment of the invention, a second folded container film 113 which is liquid-permeable, e.g. through small holes, is arranged inside the closing cap 101. This second container film can be fastened at one end to the plastic half shell and received in the first film container of the first container film 112. When the closing cap 101 is released and the flexible film tubes subsequently deploy, like nesting dolls, said film will move. In one embodiment, the container film 113 is somewhat shorter than the outer film container. The flexible tubes are not restricted in the basic shape thereof and may have other than round cross-sections. Prior to releasing the closing cap 101 from the cover plate, the container film 113 forms a folded film insert. The container film 113 forms a second film container 113a and contains a swelling agent 114, preferably comprising organic material, which while having reduced volume as such expands upon contact with liquid, and the volume thereof may be multiplied. Such swelling agents are also designated as superabsorbents and are known from incontinence products. Herein, the perforated container film with the swelling agent 114 may extend like a sealing plug inside the stoma outlet and thus further absorb liquid. Alternatively to the second film container 113a, the first film container 112a can be divided by a liquid-permeable and/or elastic container film separating an area with the swelling agent 114.

On the one hand, such steps make sure that the stoma outlet is sealed, on the other hand, liquid absorption will decrease pressure on the closing cap and thus increase sealing. In order to indicate that a replacement is required, the level of the film contents is detected via pressure or pH-value or conductivity measurement of the swelling agent 114 by means of a sensor 106. Other options are volume measurements, strain gauges, film extension or tension. The measured value can be forwarded via radio technology, such as e.g. RFID, ZIGBEE, or WLAN, to adequate receivers and therein displayed to the carrier mechanically (alarm clock, vibrator) or optically (LED lights). The signal can be configured differently depending on urgency. The closing cap may include a protrusion 115 for fastening inside the cover plate.

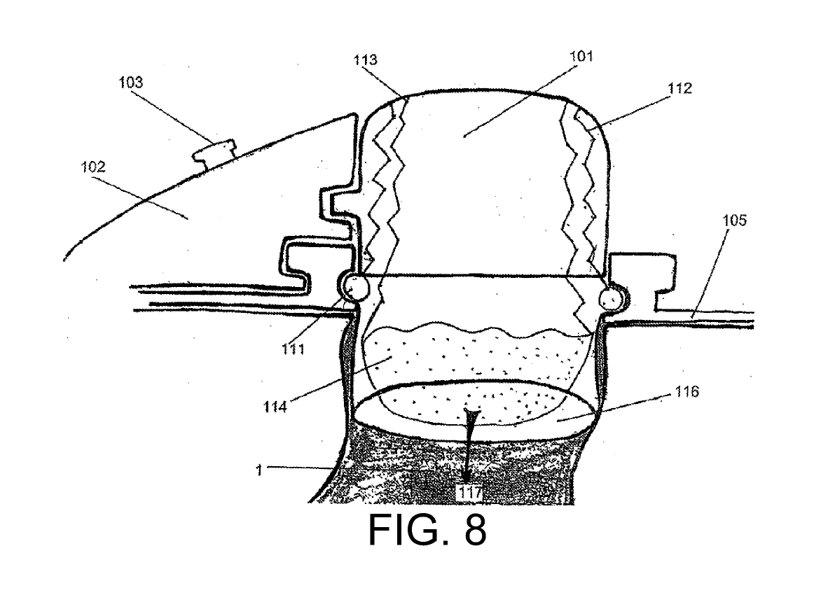

In one embodiment, represented in FIG. 8, the invention comprises an insertion aid inside the feeding bowel 1a. Said insertion aid comprises a tube 116, e.g. a soft silicone tube in the form of a cylinder, which is fasted to the base plate by one end. This tube acts as a guide for the internal film container with the organic swelling agent 114. The internal container film 113 with the swelling agent 114 moves through the tube 116 into the intestine. The direction is indicated by an arrow 117. The tube is to prevent the film container from accumulating in front of the stoma, and the closing cap 101 with the base plate 105 from being lifted up due to the increase in volume which might lead to leakage at the stoma outlet.

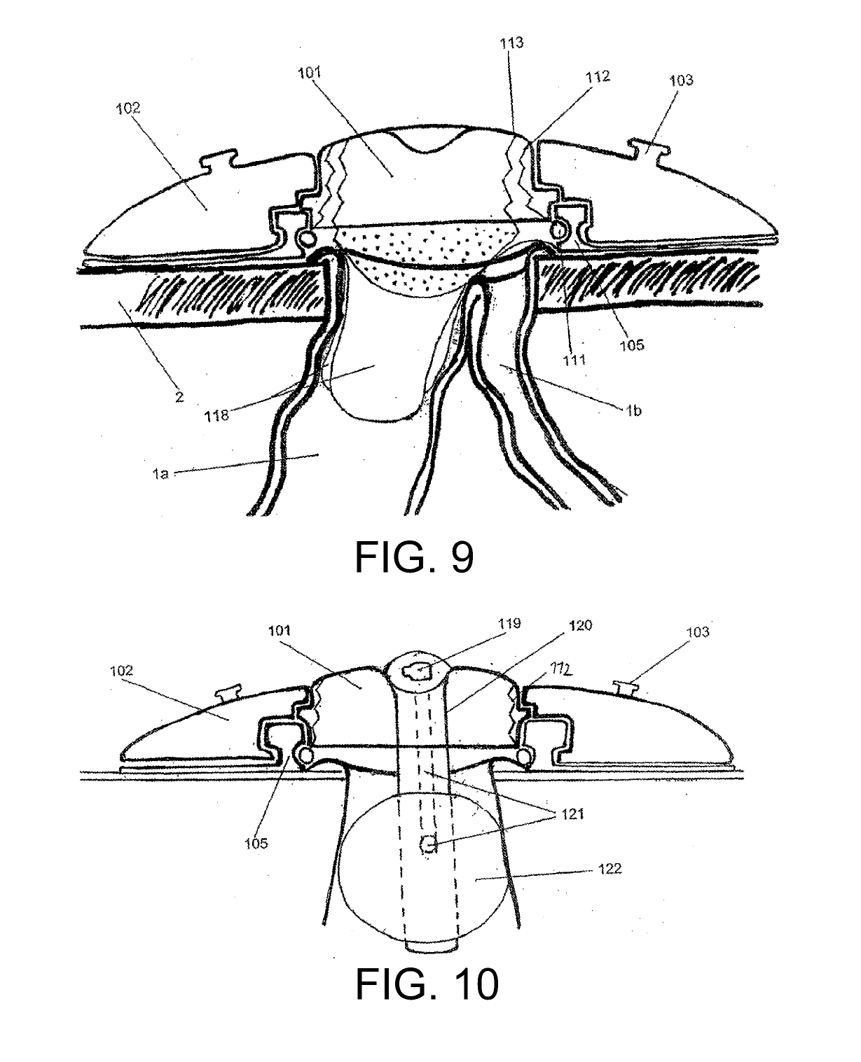

FIG. 9 shows another embodiment with an annular soft plastic canvas 118 curving into the stoma like a Bauhin's valve at the transition between the terminal ileum and caecum. Said valve is shaped so as to cause quite effective closure in the retrograde direction during replacement. Upon expansion of the film container with the swelling agent 114 the plastic canvas 118 is passively spread into the bowel. Once the full film container has been removed, the soft plastic canvas collapses again like a mucosal fold hindering uncontrolled exit of liquid from the stoma. The annular plastic canvas is fastened to the base plate 105 directly at the stoma.

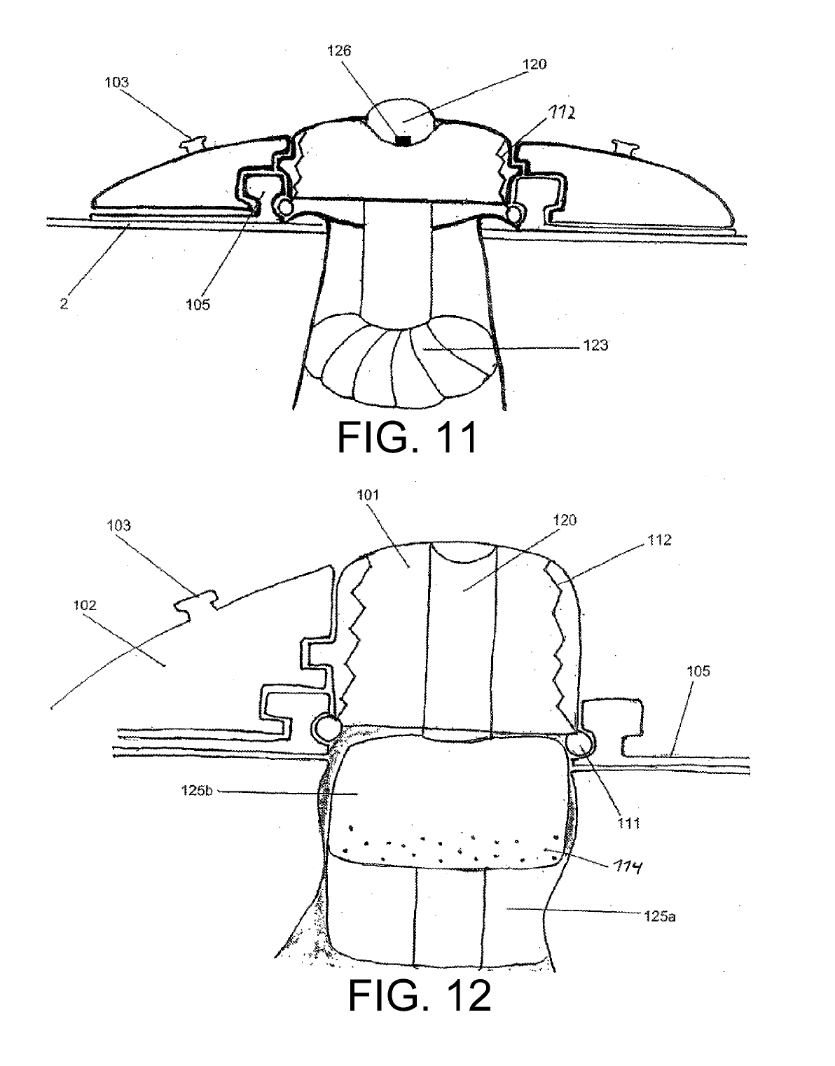

In an alternative embodiment, as represented in FIG. 11, the device comprises a connecting piece, e.g. a tube 120, preferably made of silicone, which by one end is fastened centrically to the inside of the closing cap. At the other end of the tube—located inside the intestinal lumen—an extension with an umbrella-like closure 123 is attached, which, when the closing cap has been inserted, will deploy like a curved umbrella, triggered by fluid pressure from the intestinal lumen. The umbrella rim adapts to the wall of the intestinal lumen causing some liquid retention. By means of a sensor 126 contacting the tube 120 fastened inside the closing cap and measuring the pressure applied thereto, the liquid pressure inside the intestinal lumen can thus be measured and forwarded with the indications described above. Liquid pressing past the umbrella rim on the outside is absorbed by a swelling agent, similar to the example of FIGS. 7 and 8. When a given pressure ratio (depending on the volume) is exceeded, which has been determined earlier in the measured values of the sensor, the silicone tube hanging inside the closing cap is rotated about the axis thereof, e.g. by 180°, and the film umbrella 123 closes down like an umbrella. Excessive liquid now continuing to flow is absorbed by the above-mentioned swelling agent. The closing cap 101, inside which a folded container film 112 is accommodated as in the previously mentioned examples, is removed together with the silicone tube as described above. Closure for preventing liquid from leaking out is done by means of the above-described soft plastic canvas 118.

According to FIG. 10, one embodiment the invention may comprise an inflatable balloon 122 instead of a film umbrella deploying around an extension tube, said balloon being attached to the tube 120 and operating according to the principle of balloon locking in ureter catheters, intubation tubes, etc. This balloon is inflated via a channel 121 in the tube 120 and adapts as a balloon closure to the inner side of the intestinal lumen. Liquid pressing past the balloon rim is absorbed by a swelling agent 114 in the example of FIG. 7 or 8. Once the pressure ratio has been exceeded, which is indicated as described above by a sensor 119 contacting the tube 120 fastened inside the cap and measuring the pressure applied thereto, the balloon 122 must be deaerated before the closing cap 101 is removed. This is done by opening a closure, e.g. in the form of a cap or valve (not shown) on the outside of the cap. The balloon collapses, with liquid present being absorbed via the swelling agent 114. The balloon and the closing cap together with the container film 112 can be removed with the film contents. The plastic canvasses 118 acting as the closure prevent any further liquid leakage.

In a further embodiment of the invention according to FIG. 12, a cylinder is provided which is introduced into the feeding bowel as described above for the balloon via an extension 120 arranged on the closing cap 101. The cylinder comprises two chambers. The lower chamber 125a oriented toward the intestinal lumen acts as an insertion aid and is inflatable like the balloon 122 in FIG. 10. The upper chamber 125b is meant for receiving the intestinal contents (similar to the internal film container 113 in the example of FIG. 7) and is filled with a swelling agent 114. During draining, the closing cap 101, which in the above-mentioned examples is accommodated inside a folded container film 112, is removed with the cylinder as described above to allow for disposal of the cylinder together with the swelling agent in the container film.

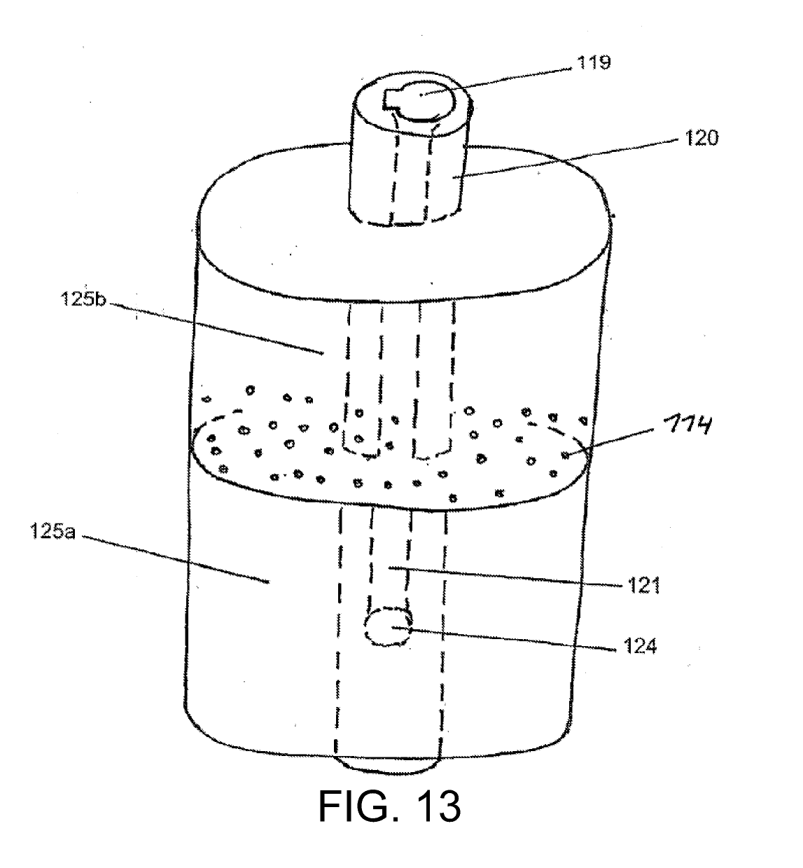

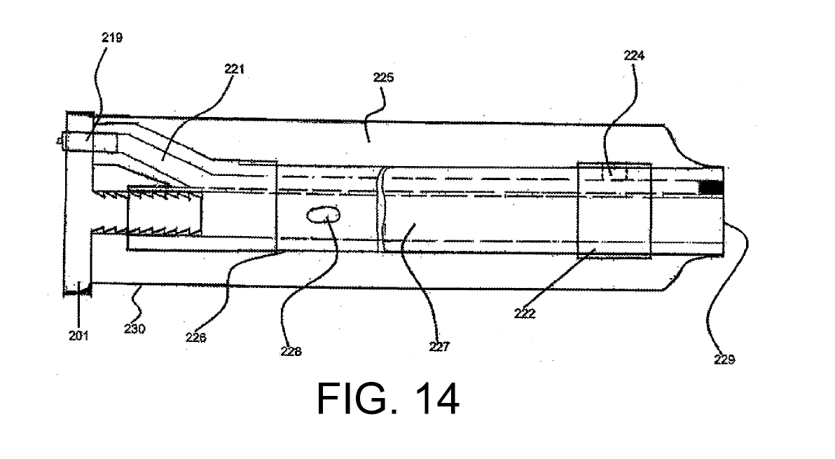

In FIG. 13, the cylindrical receptacle is represented in detail. As in the embodiment with the balloon (cf. FIG. 10), the cylinder is fastened to the closing cap via a tube 120 through which a channel for aeration and deaeration is guided. In the area of the lower chamber 125a, tube 120 has an opening 124 to the channel. At the upper end of the tube 120, an aerating and deaerating opening 119 is located. The cylindrical configuration significantly facilitates introduction into the stoma.

FIG. 14 shows an alternative sample embodiment of a device for a temporary ileostomy or urostomy closure. The length of this closing unit is adapted to the respective field of application, i.e. ileostomy or urostomy. The device comprises an open tube member protruding into the stoma and open on one side, acting as an insertion aid, and at the end portion of which, oriented towards the intestinal lumen, an inflatable volume is provided so that the tube member is fastened and inside the bowel and at the same time sealed by the inflatable volume of the stoma. Furthermore, the device comprises a chamber arranged around the tube and made for receiving the intestinal contents.

In a preferred embodiment, a modified catheter 226, e.g. made of soft silicone, is used as the tube member and fitted at the tip with an inflatable balloon 222 made of silicone. Such so-called balloon-catheters are usually used for transurethral bladder diversion. A balloon-catheter is usually made of soft silicone or latex, and is provided near the tip with a small inflatable silicone balloon which, when inflated, normally interlocks the balloon-catheter inside the urinary bladder.

The catheter 226 used in the present sample embodiment has a diameter of about 28 to 30 Charrière (approx. 9 to 10 mm). An axially extending main channel 227 of the catheter 226 connects an inlet opening 229 to an opening 228 toward a chamber 225 made as a cylinder-shaped elastic film container 230, and which is arranged around the catheter 226 and made for receiving the intestinal contents. The catheter 226 is held at one end on the inside of a closing cap 201. The inlet opening 229 at the other end of the catheter 226 opens into the intestinal lumen. The rim of the inlet opening 229 is rounded and soft. The chamber 225 is arranged around the catheter 226 and is limited by the inner side of the closing cap 201 at the stoma and laterally by an elastic container. In the sample embodiment shown, the container is formed by a cylinder-shaped elastic film container 230. This is the border to the inner lumen of the bowel and contains a swelling agent (not shown) as already described in relation to other sample embodiments. In one particular sample embodiment, the main channel 227 also contains a swelling agent.

The elastic film container 230 is preferably made of a flexible tube of thin silicone or latex sheathing the catheter 226 and the chamber 225. The elastic film container 230 is connected, e.g. glued or welded, to the catheter 226 distally from the balloon 222 like a ring near the inlet opening 229, extends over the inflatable balloon, and is connected at the proximal end to the inside of the closing cap 201, e.g. with a snap ring or wraparound ring. For this purpose, in the inner side of the closing cap 201, a recess or groove is sunk circularly, into which the proximal elastic film container can be safely anchored by a clamped connection. The film container 230 thus forms a laterally closed system. Herein, the film container 230 covers the catheter and at the distal end the inflatable balloon 222 and is welded to the catheter 226 or the main channel 229 and anchored into the closing cap 201 at the proximal end. The film container 230 is dimensioned so that before being inserted into the stoma 3, it is placed loosely around the catheter 226, and is configured to expand around the introduced catheter 226 up to the inner bowel wall when the chamber 225 is filled with liquid from the bowel. The maximum achievable diameter of the film container 230 in the bowel is also determined by the elasticity of the bowel wall.

In this sample embodiment, opening 228 is oval and attached near the closing cap 201. In one sample embodiment, opening 228 in the main channel 227 is provided with a membrane (not shown), which as a one-way opening like a check valve only allows liquid from the main channel 227 to flow into the chamber 225. In case of a change in body position of the carrier, the membrane is to close the opening 228 between the chamber 225 and the main channel 227 and prevent reflux from the chamber 225 into the main channel 227.

Inside the catheter 226, a balloon channel 221 connects a port 219 on the closing cap 201 to the balloon 222. The balloon channel 221 opens into the balloon 222 inside an opening 224 in the wall of the balloon channel 221. Via the port 219 and the balloon channel 221, the balloon 222 can be filled with air or liquid by means of an injection device, e.g. an injection syringe, for example with a volume of about 5 to 10 ml. Thereby, the catheter 226 is held inside the bowel sealing the same from the stoma 3 in the location of the expanding balloon 222. In some sample embodiments, port 219 is fitted with a check valve, which in special sample embodiments is made to be an unlockable check valve. The check valve keeps the balloon 222 full as long as the catheter 226 is to stay inside the bowel. When the check valve is unlocked at the port 219, e.g. by pushing a tool made as a push-button, the filling is released from the balloon 222, and the catheter is no longer held by the balloon 222. The balloon channel 221 is closed near the inlet opening 229.

For use, the carrier introduces said inventive device via the catheter 226 into the stoma. Once the catheter 226 has been inserted to sufficient depth, the carrier places the closing cap 201 into the cover plate 102 locking it therein. Next, the carrier inflates the balloon 222, e.g. with a syringe. The inflated balloon 222 holds the catheter 226 inside the bowel and prevents intestinal liquid from flowing past the catheter 226 on the outside.

As long as the catheter 226 is thus inserted, intestinal liquid penetrates trough the inlet opening 229 from the bowel into the main channel 227 of the catheter 226. Then, intestinal liquid flows through the opening 228 from the main channel 227 into the chamber 225 surrounded by a film container 230. A swelling agent inside the film container 230 receives the intestinal liquid, and the film container 230 dilates. In one example, the swelling agent binds the intestinal liquid inside the chamber 225 in a jelly-like form. Thus, intestinal liquid once absorbed will stay inside the chamber 225 while being permanently bound, thereby facilitating subsequent disposal. Due to a capillary effect, the swelling agent further supports the absorption of intestinal liquid inside the chamber 225 and the expansion of the film container 230, whereby more liquid can be received inside the chamber 225. Furthermore, due to the capillary effect of the swelling agent, intestinal liquid is received inside the chamber 225 even with low internal pressure of the bowel.

In a further sample embodiment, inside the closing cap 201, a rinsing port (not shown) is arranged, which is also provided with a simple check valve and extends from a rinsing channel towards the main channel 227. The rinsing channel opens between the closing cap 201 and the opening 228 into the main channel 227. As required, a rinsing liquid, e.g. water or NaCl solution, is injected by a syringe into the main channel 227 via the rinsing channel. The rinsing liquid is to keep the main channel 227 permeable, in particular it is to eliminate possible conglutinations of fiber material, protein flakes, etc. Such solid components are diluted by the NaCl solution introduced and washed into the intestinal lumen. In one particular sample embodiment, the opening 228 is closed before the rinsing liquid is injected into the main channel 227. For this purpose, e.g. a slide closes the opening 228 when the syringe is introduced into the rinsing port. Thereby, during rinsing, rinsing liquid is prevented from escaping through the opening 228 into the chamber 225.

In another sample embodiment, a sensor for measuring the level at the catheter 226 or inside the chamber 225 is provided, which transmits a signal to a corresponding receiver, e.g. in the cover plate 102, before the film container is completely full. The sensor is for instance a pressure sensor triggered by the rise of the swelling agent, or any other of the sensors mentioned above. As described above, the carrier then receives the measured value or a recommendation for replacing the closing cap 201 with the catheter 226 and the film container 230 together with the contents.

In order to remove the closing cap 201 together with the attached film container 230 and contents, the balloon 222 is first deaerated via port 219. For this purpose and if required, the check valve can be unlocked by means of a syringe, or by implementing the above-mentioned push-button unlocking the valve. In one sample embodiment, the carrier releases the closing cap 201 from the cover plate 102 and extracts the film container 230. The swelling agent inside the film container is holding the liquid inside the film container 230. A container film, such as the container film 112 of FIG. 7, which is accommodated inside the closing cap 101 and held in the cover plate 102, can be provided in addition and surrounds the film container 230 during extraction.

In another sample embodiment, such a container film (not represented) is attached only shortly before the catheter 226 is removed. For this purpose, a separate container film is made as an accordion-pleated film container. Once the balloon 222 has been deaerated, the container film with an open end is clamped on the snap-in ring 103 of the cover plate 102. The container film is made so that the carrier may seize the grip 104 on the closing cap 201 through the container film. For this purpose, the container film is made to be either thin or flexible or even provided with a hole through which the closing cap 201 can be seized. Possibly, the edge of the hole can be glued all around with the closing cap so that no intestinal liquid may escape. The carrier releases the closure of the closing cap 201 above the grip 104 and extracts the catheter 226 with the film container from the inside of the bowel. The container film completely surrounds the closing cap 201 and the film container 230 with the entire contents. Thus, the container film allows for contactless and clean disposal.

When the contents has been removed, the stoma 3 is briefly open and only provided with the cover plate 102. Now, the carrier may either place another closing cap 201 with another catheter 226 or a stoma pouch on the snap-in ring of the cover plate 102.

As with replacement of a stoma pouch, when the closing cap 201 is replaced, only little intestinal contents should emerge from the stoma 3. When changing stoma pouches, it is usually sufficient to dab the stoma 3 with a pad for the liquid to be absorbed.

Another embodiment of the invention, not shown, comprises at least one, preferably two spring-mounted half discs fastened at the upper edge of the base plate. When the closing cap is introduced into the cover plate, both half discs will yield approximately in parallel to the abdominal wall, respectively into the associated half of the cover plate cavity. Due to the simple spring mechanism, said half discs will close when the closing cap is removed and form some kind of valve barrier across the stoma outlet. This is meant as additional safety from intestinal contents leakage during replacement.

The sample embodiments of the inventive device provide practical resources for patients with ileostomy or urostomy wherein the ureter has to be discharged via an ileum conduit or colon conduit. It is to enable omission of a conventional stoma pouch during a limited period of time. An artificial bowel or bladder outlet strongly affects social life as well as intimate private life of the person involved. Due to tabooing of urine and stool related subjects, frequently shame and inferiority complexes occur. The invention provides a temporary cover of a stoma, which is configured so that fluid emerging from the cover during replacement is collected in a receptacle or by being introduced into the intestinal lumen will already absorb fluid therein in a receptacle. With the sample embodiments of the inventive device, some relief in mastering such drastic changes is obtained, and thus self-confidence, and quality of life, and mobility of the person involved are improved. Furthermore, the sample embodiments of the invention create an economical and easy-to-use device enabling a person involved, e.g. having an ileostomy/urostomy treatment, to go without the required pouch supply for a certain time. Furthermore, the sample embodiments of the invention enable secure stoma treatment to be ensured, relieving the stoma carrier from the “strategy of secrecy about otherness”, providing the person involved with a new sense of self-worth and making him or her socially acceptable.

The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A device for a temporary seal of a stoma coupled to an intestine having an intestinal lumen, comprising:
(a) a base plate configured to be applied permanently about a periphery of the stoma on an outside of a body;
(b) a cover that is releasably connected to a base plate for forming a temporary stoma steal and that is configured in such a way that it forms a volume for receiving at least one receptacle for fluid emerging from the stoma; and
(c) a tube-shaped container that is detachably coupled with the base plate and disposed within the cover, but not attached to the cover, the container configured to be separable from the base plate, the container opening to the stoma and configured to receive fluid emerging therefrom.

2. The device according to claim 1, wherein the receptacle is also releasably coupled with the base plate and wherein the cover is releasable from the base plate separately from the receptacle.

3. The device according to claim 1, wherein the base plate includes a recess into which the receptacle can be hung at the open end thereof.

4. The device according to claim 1, wherein the cover is made to be cup or half shell shaped.

5. The device according claim 1, wherein the cover is formed of a closing cap and a cover plate coupling the closing cap in an opening of the cover plate to the base plate via the opening of a stoma.

6. The device according to claim 1, wherein the cover is connected via a connecting piece to an umbrella-like member extending into the bowel and which is configured to deploy at the end opposite the cover of the connecting piece and hinder liquid leakage, under internal intestinal pressure or influence of a carrier.

7. The device according to claim 1, wherein the device comprises an inflatable balloon introduced into the stoma, which is configured to be aerated and de-aerated from the outside in order to optionally seal the stoma.

8. The device according to claim 1, wherein on the inside of the cover, a cylindrical receptacle protruding into the urostomy is provided comprising a first chamber oriented towards the intestinal lumen and made to be inflatable, and comprising a second chamber adjacent to the first chamber and containing the swelling agent for receiving the contents emerging from the urostomy.

9. The device according to claim 1, wherein the cover and/or the base plate are at least partially coated with a material opposing adherence of dirt particles and/or formation of odor.

10. The device according to claim 1, wherein the cover plate or the base plate has at least one disc which is biased and made for closing the opening under the effect of spring force across the stoma when no closing cap is inserted.

11. A device for a temporary ileostomy or urostomy closure, comprising:
(a) a base plate to be permanently attached in the periphery of a stoma on the outside of a body surface; and
(b) a cover that is releasably connected to the base plate for forming a temporary stoma closure, wherein on the inner side of the cover, a tube member protruding into the stoma is fastened, and wherein a chamber is made around the tube member, which is configured for receiving contents emerging from the stoma through an axial main channel of the protruding tube member.

12. The device according to claim 11, wherein the main channel is configured for receiving intestinal liquid via an inlet opening, and which is connected to the chamber via an opening which is permeable for liquid only in the direction of the main channel into the chamber.

13. The device according to claim 11, wherein the chamber and/or the main channel is/are filled with a swelling agent for absorbing liquid.

14. The device according to claim 11, wherein the chamber is made of an elastic film container surrounding the tube member.

15. The device according to claim 11, wherein the tube member comprises a balloon which when inflated is configured to fix the tube member inside the stoma and/or seal the stoma from the outside.

16. The device according to claim 11, wherein the main channel is configured to be rinsed via a rinsing port inside the closing cap and a rinsing channel.

17. The device according to claim 11, wherein the tube member is a catheter which is formed at the top with an integrated inflatable balloon.

18. The device according to claim 11, wherein the device comprises a sensor unit which is configured to indicate the necessity of replacement based on a sensor value.

19. The device according to claim 11, further comprising a container film which is configured to be attached to a recess of the base plate, to allow for loosening of the closing cap, and to receive the device therein when it is removed from the stoma.
